(19) United States
(12) Patent Application Publication
(10) Pub. No.: US 2010/0137916 A1
(43) Pub. Date: Jun. 3, 2010

(54) SPINAL PLATES FOR STABILIZING SEGMENTS

(75) Inventors: Richard A. Hynes, Melbourne, FL (US); Gary S. Lindemann, Collierville, TN (US); William David Armstrong, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., An Indiana Corporation, Warsaw, IN (US)

(21) Appl. No.: 12/327,417

(22) Filed: Dec. 3, 2008

(51) Int. Cl. A61B 17/70; A61B 17/04

(52) U.S. Cl. 606/280; 606/286; 606/301

(57) ABSTRACT

In some aspects, the spinal plates of the present disclosure are sized and shaped for use in a spinal level adjacent to a previously treated spinal level. The spinal plates of the present disclosure are generally no-profile or low-profile plates. That is, the spinal plates are sized and shaped for positioning entirely within the disc space between adjacent vertebrae such that the plates either do not extend beyond the outer boundaries of the vertebrae (no profile) or extend only slightly beyond the outer boundaries of the vertebrae (low profile). The spinal plates of the present disclosure are also configured to receive fixation members, such as bone screws, in a hyper-angulated orientation. The hyper-angulated screws facilitate optimal cortical bone purchase or penetration of the adjacent vertebrae to fixedly secure the spinal plates to the vertebrae.

### BACKGROUND

The present disclosure generally relates to orthopedic implants and systems for correction of spinal injuries and/or deformities, and more specifically, but not exclusively, concerns implants and systems for stabilizing a portion of the spine to allow correction and/or healing thereof. In some embodiments, the present disclosure is directed to improved apparatus, systems, and assemblies for stabilizing vertebrae.

Currently, in some instances the standard of care for treating spinal injuries and deformities, such as tumors, trauma, degenerative disc disease, etc., is a discectomy with interbody fusion. In some instances, supplemental fixation elements are also utilized to further stabilize the vertebrae to encourage fusion. For example, spinal plates may be securely attached to the vertebrae. In some instances, after fusion of a first spinal level, an adjacent spinal level may require treatment.

Accordingly, in some aspects, the present disclosure relates to spinal plates for use in stabilizing a spinal level adjacent to a previously treated spinal level. Alternatively, the spinal plates of the present disclosure are utilized to treat a spinal level such that a subsequent treatment of an adjacent spinal level is not inhibited. In some instances, the spinal plates of the present disclosure are sized and shaped for use in a level adjacent to a previously implanted stabilization device, such as an intervertebral disc and/or a spinal plate. In that regard, in some embodiments the spinal plates of the present disclosure are no or low profile plates. That is, the spinal plate is sized and shaped for positioning entirely within the disc space between adjacent vertebrae such that it does not extend beyond the outer boundaries of the vertebrae (no profile) or extends only slightly beyond the outer boundaries of the vertebrae (low profile). In some embodiments, the spinal plates of the present disclosure are configured to receive hyper-angulated screws. In some instances, the hyper-angulated screws facilitate optimal cortical bone purchase or penetration for fixedly securing the spinal plates to the vertebrae.

Therefore, there remains a need for improved apparatus, systems, and assemblies for stabilizing the spinal column.

### SUMMARY

In one embodiment, the present disclosure provides a spinal plate for use in stabilizing a spinal segment. In some instances the spinal plate includes openings extending therethrough to receive fixation members in a hyper-angulated orientation.

In another embodiment, the present disclosure provides a spinal plate for positioning between a first vertebra and a second vertebra. The spinal plate comprises a generally rectangular body portion. The body portion includes a first elongated engagement surface for fixedly engaging the first vertebra and a second elongated engagement surface opposite the first engagement surface for fixedly engaging the second vertebra. The second engagement surface extends substantially parallel to the first engagement surface. The first and second engagement surfaces are separated by a first height. A first axis extends substantially perpendicular to the first and second engagement surfaces. A first sidewall extends between and substantially perpendicular to the first and second engagement surfaces. A second sidewall extends between and substantially perpendicular to the first and second engagement surfaces opposite the first sidewall. The second sidewall extends substantially parallel to the first sidewall. The first and second sidewalls are separated by a first width. The first width is less than the first height. A first substantially cylindrical bore extends from the first sidewall to the second sidewall through the body portion at an oblique angle of at least 30 degrees with respect to the first axis. The first bore is sized to receive and mate with a bone fixation device for securing the body portion to the first vertebra.

In another embodiment, the present disclosure provides a spinal implant for stabilizing a pair of adjacent vertebrae without penetrating a sidewall of the vertebrae. The spinal implant comprises a central portion extending along a first plane, a first engagement portion extending from an upper part of the central portion, and a second engagement portion extending from a lower part of the central portion. The first engagement portion extends along a second plane that is at a first oblique angle with respect to the first plane. The second engagement portion extends along a third plane that is at a second oblique angle with respect to the first plane and substantially perpendicular to the second plane. A first opening extends through the first engagement portion substantially perpendicular to the second plane. The first opening is sized and shaped to receive and mate with a first bone fixation device for securing the first engagement portion to one of the adjacent vertebrae. A second opening extends through the second engagement portion substantially perpendicular to the third plane. The second opening is sized and shaped to receive and mate with a second bone fixation device for securing the second engagement portion to the other of the adjacent vertebrae.

In another embodiment, a method of stabilizing a first vertebra and a second vertebra adjacent to a previously stabilized spinal level that includes the first vertebra is disclosed. The method comprises providing a prosthetic device sized to fit substantially within a disc space between the first and second vertebra, gaining access to the disc space, and inserting the prosthetic device into the disc space in a low or no profile orientation with respect to the first and second vertebra. The prosthetic device extends within the disc space less than ⅓ of the length of the vertebral bodies of the first and second vertebra after insertion. A first bone anchor is extended through a first bore in the prosthetic device and engaged with an endplate of the first vertebra. The first bone anchor extends at an angle of approximately 45 degrees relative to a central axis of the prosthetic device. A second bone anchor is extended through a second bore in the prosthetic device and engaged with an endplate of the second vertebra. The second bone anchor also extends at an angle of approximately 45 degrees relative to the central axis the prosthetic device such that the first bone anchor and the second bone anchor extend substantially perpendicular to one another.

These and other aspects and advantages of the present disclosure will become apparent to those skilled in the art from the detailed description of the embodiments set forth below.

### BRIEF DESCRIPTION OF THE DRAWINGS

FIG. 1 is a diagrammatic side view of an arrangement that embodies aspects of the present disclosure.

FIG. 2 is a diagrammatic front view of a prosthetic device according to one aspect of the present disclosure.

FIG. 3 is a diagrammatic side view of the prosthetic device of FIG. 2.

FIG. 4 is a diagrammatic cross-sectional side view of the prosthetic device of FIG. 2 taken along section line 4-4.

FIG. 5 is a diagrammatic cross-sectional side view of the prosthetic device of FIG. 2 taken along section line 5-5.

FIG. 6 is a diagrammatic side view of the prosthetic device of FIG. 2 engaged with a pair of vertebrae according to one embodiment of the present disclosure.

FIG. 7 is a diagrammatic front view of a prosthetic device according to another aspect of the present disclosure.

FIG. 8 is a diagrammatic side view of the prosthetic device of FIG. 7.

FIG. 9 is a diagrammatic front view of a prosthetic device according to another aspect of the present disclosure.

FIG. 10A is a diagrammatic side view of the prosthetic device of FIG. 9.

FIG. 10B is a diagrammatic side view of the prosthetic device of FIG. 9 engaged with a pair of vertebrae according to one embodiment of the present disclosure.

FIG. 11 is a diagrammatic top view of the prosthetic device of FIG. 9.

FIG. 12 is a diagrammatic front view of a prosthetic device according to another aspect of the present disclosure.

FIG. 13 is a diagrammatic side view of the prosthetic device of FIG. 12.

FIG. 14 is a diagrammatic perspective view of a prosthetic device according to another aspect of the present disclosure.

FIG. 15 is a diagrammatic side view of the prosthetic device of FIG. 14 engaged with a pair of vertebrae according to one embodiment of the present disclosure.

FIG. 16 is a diagrammatic front view of the prosthetic device of FIG. 14 engaged with a pair of vertebrae according to one embodiment of the present disclosure.

FIG. 17 is a diagrammatic perspective view of a prosthetic device according to another aspect of the present disclosure.

FIG. 18 is a diagrammatic front view of the prosthetic device of FIG. 17.

FIG. 19 is a diagrammatic side view of the prosthetic device of FIGS. 17 and 18.

FIG. 20 is a diagrammatic front view of the prosthetic device of FIGS. 17, 18, and 19 engaged with vertebrae of spinal column according to one embodiment of the present disclosure.

FIG. 21 is a diagrammatic side view of the prosthetic device of FIGS. 17, 18, and 19 engaged with the vertebrae of the spinal column according to one embodiment of the present disclosure.

FIG. 22 is a diagrammatic perspective view of a prosthetic device according to another aspect of the present disclosure.

### DETAILED DESCRIPTION

For the purposes of promoting an understanding of the principles of the present disclosure, reference will now be made to the embodiments illustrated in the drawings and specific language will be used to describe these embodiments. It is nevertheless understood that no limitation of the scope of the disclosure is thereby intended. Modification of the disclosed embodiments and/or further application of the principles of the present disclosure are fully contemplated as would occur to one skilled in the art to which the present disclosure relates.

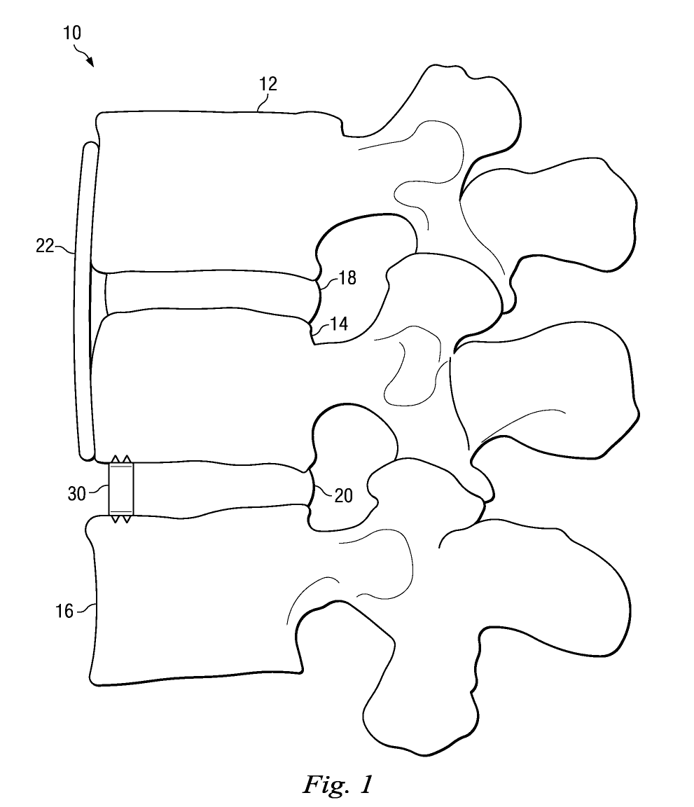

Referring now to FIG. 1, shown therein a diagrammatic side view of an arrangement 10 that embodies aspects of the present disclosure. In particular, the arrangement 10 includes three adjacent vertebra 12, 14, and 16 separated by intervertebral discs 18 and 20. Generally, the vertebra 12 and 14 with disc 18 may be considered a first spinal level, while the vertebra 14 and 16 with disc 20 may be considered an adjacent second spinal level. The first spinal level has been stabilized with a spinal plate 22. As shown, the plate 22 extends across a majority of the anterior faces of the vertebra 12 and 14. This prevents a similar plate from being utilized to stabilize the adjacent spinal level consisting of vertebra 14 and 16 because there is insufficient room to mount another plate onto vertebra 14. Thus, in some instances a prosthetic device 30 according to one embodiment of the present disclosure is utilized to stabilize the adjacent spinal level. In that regard, the prosthetic device 30 is sized and shaped for use in a spinal level adjacent to a previously implanted stabilization device, such as an intervertebral disc and/or a spinal plate. Further, the prosthetic device 30 is sized to have a no or low profile. That is, the prosthetic device 30 is sized and shaped for positioning entirely within the disc space between the vertebrae 14 and 16 such that it does not extend beyond the anterior boundaries of the vertebrae (no profile) or extends only slightly beyond the anterior boundaries of the vertebrae (low profile).

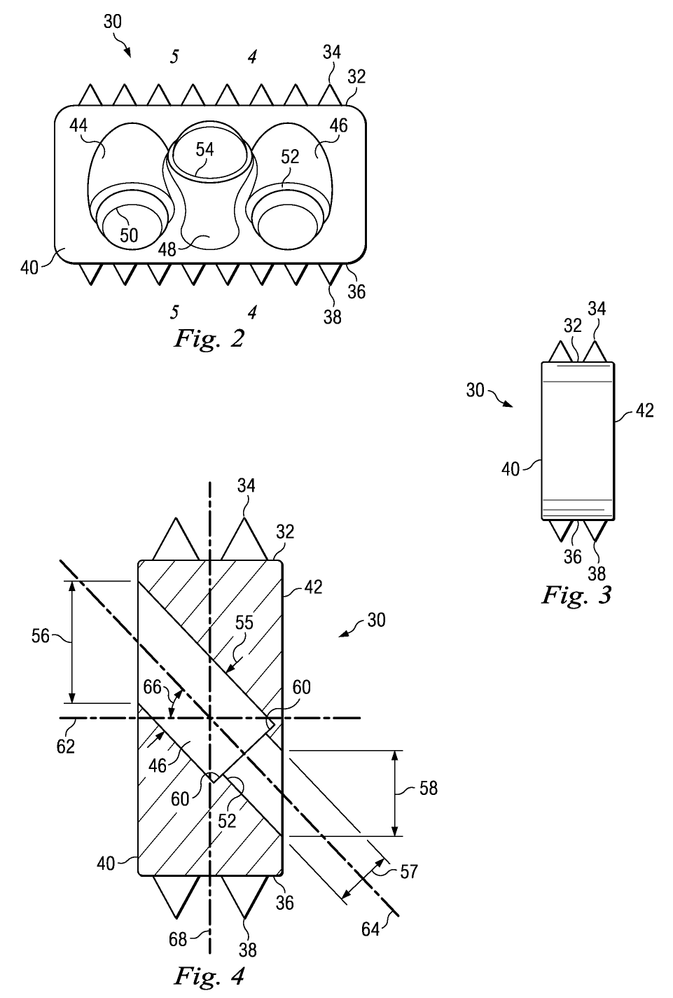

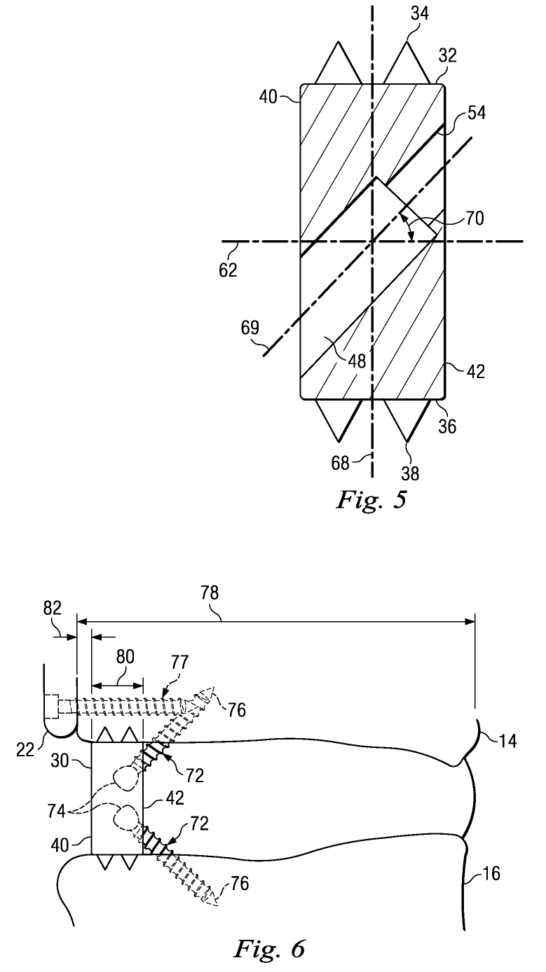

Referring now to FIGS. 2-6, shown therein are various illustrations of the prosthetic device 30 according to one embodiment of the present disclosure. In particular, FIG. 2 is a diagrammatic front view of the prosthetic device 30; FIG. 3 is a diagrammatic side view of the prosthetic device 30; FIG. 4 is a diagrammatic cross-sectional side view of the prosthetic device 30 taken along section line 4-4 of FIG. 2; FIG. 5 is a diagrammatic cross-sectional side view of the prosthetic device 30 taken along section line 5-5 of FIG. 2; and FIG. 6 is a diagrammatic side view of the prosthetic device 30 engaged with a pair of vertebrae.

Referring more specifically to FIGS. 2 and 3, in the present embodiment the prosthetic device 30 has a generally rectangular profile and includes an upper surface 32 for engaging with an upper vertebra. The upper surface 32 has a plurality of bone engagement features 34 protruding therefrom. Similarly, opposite the upper surface 32 the prosthetic device 30 includes a lower surface 36. The lower surface 36 also has a plurality of bone engagement features 38 protruding therefrom. In the present embodiment, each of the bone engagement features 34, 38 comprises a spike for penetrating the end plate of an adjacent vertebra. In other embodiments, the bone engagement features comprises other structures and features for engaging bone. For example, in some instances the upper and/or lower surfaces include a single elongated protrusion or keel for engaging a prepared recess or opening in the adjacent vertebra. In some instances, the upper and/or lower surfaces include projections, such as spikes, keels, ridges, or other surface textures; surface treatments, such as chemical etching, bead-blasting, sanding, grinding, serrating, diamond-cutting, coating with a biocompatible and osteoconductive material (such as hydroxyapatite (HA), tricalcium phosphate (TCP), or calcium carbonate), or coating with osteoinductive materials (such as proteins from the transforming growth factor (TGF) beta superfamily or bone-morphogenic proteins, such as BMP2 or BMP7); or other features for enhancing engagement with the surrounding bone structures.

The prosthetic device 30 also includes an anterior surface 40 and an opposing posterior surface 42. In the present embodiment, three openings 44, 46, and 48 extend through the prosthetic device 30 from the anterior surface 40 to the posterior surface 42. In that regard, each of the openings 44, 46, and 48 is a generally cylindrical bore sized to receive a bone fixation device for securing the prosthetic device 30 to the adjacent vertebra. In other embodiments, the openings 44, 46, and 48 have other geometrical profiles for receiving and/or mating with bone fixation members. In some embodiments, one or more of the openings 44, 46, and 48 has a different profile than one or more of the other openings 44, 46, and 48. In the present embodiment, the opening 48 is substantially centered about a midline or midpoint of the prosthetic device 30, while the openings 44 and 46 are positioned laterally on each side of the opening 48.

The cylindrical openings 44, 46, and 48 of the illustrated embodiment include a seat or collar 50, 52, and 54, respectively. Generally, each of the collars 50, 52, and 54 define a cylindrical bore having a reduced diameter relative to the majority of the corresponding opening 44, 46, and 48. For example, referring more specifically to FIG. 4, the majority of the opening 46 has a diameter 55 such that adjacent the anterior surface 40 the opening 46 has a height 56, while adjacent the collar 52 the opening 46 has a reduced diameter 57. Accordingly, adjacent the posterior surface 42 the opening 46 has a height 58 that is less than the height 56. The reduced diameter 57 of the opening adjacent the posterior portion of the prosthetic device 30 defines an annular surface 60 for limiting the translation of a bone fixation member posteriorly through the opening 46. In that regard, in some instances the surface 60 is sized and shaped to mate with a head portion of a multi-axial bone screw or other bone anchor.

An axis 62 extends substantially perpendicular to the anterior and posterior surfaces 40, 42 of the device 30. The opening 46 extends along an axis 64 that is at an oblique angle 66 with respect to the axis 62. Accordingly, the opening 46 extends from an upper portion of the anterior surface 40 to a lower portion of the posterior surface 42 along the axis 64. In the present embodiment the axis 64 extends at an oblique angle 66 of approximately 45 degrees with respect to the axis 62. Generally, the oblique angle 66 is between about 20 degrees and about 70 degrees, and in some instances is between about 30 degrees and about 60 degrees. In some embodiments, the oblique angle 66 of the axis 64 relative to the axis 62 is selected to allow insertion of the bone fixation devices through the prosthetic device 30 to facilitate engagement of the bone fixation devices with cortical bone of an adjacent vertebra. In that regard, the prosthetic device 30 is considered suitable for use with hyper-angulated bone fixation devices, and in some embodiments hyper-angulated bone screws. Generally, in the context of the present disclosure hyper-angulated bone fixation devices, anchors, and/or screws are considered to be those that are configured to extend at an oblique angle greater than 25 degrees with respect to the axis 62 when received within the prosthetic device 30 and engaged with the adjacent bone structure.

Referring more specifically to FIG. 5, in some aspects the opening 48 is substantially similar to the opening 46 described above. For example, the collar 54 of the opening 48 has a reduced diameter relative to the majority of the opening and the collar 54 defines an annular surface for limiting the translation of a bone fixation member posteriorly through the opening 48. However, the opening 48 extends from an lower portion of the anterior surface 40 to an upper portion of the posterior surface 42, whereas openings 44 and 46 extend from an upper portion of the anterior surface 40 to a lower portion of the posterior surface 42. In that regard, an axis 68 extends substantially perpendicular to the upper and lower surfaces 32, 36 of the device 30. The opening 48 extends along an axis 69 that is at an oblique angle 70 with respect to the axis 68. In the present embodiment the axis 69 extends at the oblique angle 70 of approximately 45 degrees with respect to the axis 62. Generally, the oblique angle 70 is between about 20 degrees and about 70 degrees, and in some instances is between about 30 degrees and about 60 degrees. In some embodiments, the oblique angle 70 of the axis 69 relative to the axis 68 is selected to allow insertion of the bone fixation devices through the prosthetic device 30 to facilitate engagement of the bone fixation devices with cortical bone of an adjacent vertebra. In that regard, the opening 48 is suitable for use with hyper-angulated bone fixation devices, and in some embodiments hyper-angulated bone screws. In some instances, the hyper-angulated screws facilitate optimal cortical bone purchase or penetration for fixedly securing the prosthetic device 30 to the adjacent vertebrae.

In the present embodiment, the opening 48 extends substantially perpendicular to the opening 46, as illustrated by FIGS. 4 and 5. Accordingly, in embodiments such as the illustrated embodiment where the opening 44 and the opening 46 extend substantially parallel to one another, the opening 48 extends substantially perpendicular to both of the openings 44 and 46. In other embodiments, the opening 48 extends at an oblique angle with respect to one or both of the openings 44 and 46. In that regard, in some instances, the openings 44 and 46 do not extend substantially parallel to one another. In some embodiments, the openings 44 and 46 extend at different angles to accommodate patient anatomy, previously implanted surgical devices (e.g., bone screw, intervertebral disc, etc.), or other considerations that make it desirable to have the openings 44 and 46 extending non-parallel to one another. Generally, however, the opening 48 extends at an angle between about 40 degrees and about 140 degrees relative to the openings 44 and 46.

As described above, the opening 48 is substantially centered, while the openings 44 and 46 are positioned laterally on each side of the opening 48. In that regard, in some instances when the prosthetic device 30 is positioned within the spinal column the opening 48 is utilized to introduce a bone fixation device into the midline of a vertebra, while the openings 44 and 46 are utilized to introduce bone fixation devices in an offset configuration, spaced from the midline of the vertebra. Accordingly, in some instances the opening 48 is utilized to secure the prosthetic device 30 to a vertebra that has previously received bone anchors or screws in an offset configuration. Similarly, the openings 44 and 46 are utilized to secure the prosthetic device 30 to a vertebra that has previously received bone anchors or screws in a midline configuration. In that regard, while the opening 48 is illustrated as allowing the introduction of the midline bone fixation device into an upper vertebra adjacent the upper surface 32 and the openings 44 and 46 are illustrated as allowing the introduction of the offset bone fixation devices into a lower vertebra adjacent the lower surface 36, it fully contemplated that these orientations are reversed in other embodiments, such that the midline bone fixation device is introduced into the lower vertebra and the offset bone fixation devices are introduced into the upper vertebra.

Referring more specifically to FIG. 6, the prosthetic device 30 has been secured to vertebra 14 and 16 by bone anchoring devices 72. Each bone anchoring device includes a head portion 74 and a threaded bone engaging portion 76. A bone anchoring device 72 has been inserted into each of the openings 44, 46, and 48 such that the head portion 74 of the bone anchoring device 72 mates with the collars 50, 52, and 54 of the openings and the threaded bone engaging portion 76 engages the cortical bone of the adjacent vertebra 14 and 16. In that regard, the plate 22 is illustrated as being secured to the vertebra 14 with a pair of offset bone screws, of which bone screw 77 is shown. As illustrated, the midline positioning of the bone anchoring device 72 received within opening 48 allows the prosthetic device 30 to be secured to the vertebra 14 without interfering with the plate 22 or bone screw 77. Accordingly, in this manner the prosthetic device 30 is suitable for use in the spinal level adjacent a previously treated spinal level. In an alternative embodiment, the plate 22 is secured to the vertebra 14 via a midline bone screw. In such an alternative embodiment, the prosthetic device 30 is turned over such that the lower surface 36 engages vertebra 14 and the openings 44 and 46 are utilized to engage a pair of bone anchoring device 72 with vertebra 14 in an offset configuration so as not to disturb the midline bone screw of plate 22.

As shown in FIG. 6, the vertebral bodies or endplates of the vertebra 14 and 16 have a width 78 between an anterior boundary and a posterior boundary. Depending on the patient, the region of the spine, and other factors the width 78 is generally between about 15.0 mm and about 40.0 mm, but in some instances may be outside of these ranges. In the illustrated embodiment, the width 78 is approximately 26.0 mm. The prosthetic device 30 has a thickness 80 between the anterior surface 40 and the posterior surface 42. Generally, the thickness 80 of the prosthetic device 30 is between about 2.0 mm and about 10.0 mm. In the present embodiment, the thickness 80 is about 7.5 mm. Accordingly, the prosthetic device 30 may be positioned entirely within the outer anterior and posterior boundaries of the vertebrae 14 and 16. In the present embodiment, the prosthetic device 30 is shown spaced from the anterior boundary of the vertebrae 14 and 16 by a distance 82. In that regard, the distance 82 represents the distance from the anterior surface 40 of the prosthetic device 30 to the anterior most boundary of the vertebrae 14 and 16. Since typical vertebrae are contoured such that they taper distally as they extend laterally from the anterior midline of the vertebra, the anterior surface 40 is positioned closer to the anterior boundary of the vertebrae. In some instances, the anterior surface 40 extends beyond the anterior boundary of the vertebrae in at least one area. In such instances, the prosthetic device 30 is considered to be implanted in a low profile orientation. Where the prosthetic device 30 is positioned entirely within the boundaries defined by the vertebrae, it is considered to implanted in a no profile orientation.

The combination of the low or no profile orientation of the prosthetic device 30 along with the hyper-angulated openings 44, 46, and 48 facilitates a greater capture of cortical bone compared to traditional anterior plates that engage the cortical wall of the vertebrae, rather than the vertebral endplates. In that regard, the prosthetic devices of the present disclosure facilitate the use of longer bone screws that, in turn, create greater cortical bone engagement as they extend through the vertebral body via the hyper-angulated approach. Also, the combination of the low or no profile orientation of the prosthetic device 30 along with the hyper-angulated openings 44, 46, and 48 limits or eliminates impingement or coverage of the anterior longitudinal ligament, which results in less anatomic compromise and reduces the likelihood of an adjacent level being affected by calcification. In that regard, the prosthetic devices of the present disclosure are also utilized in some instances to decrease stress and/or forces across adjacent discs by providing a more localized and concentrated stiffness compared to typical anterior plates. Also, the prosthetic devices of the present disclosure are able to share loads and/or stresses imparted upon the vertebrae because the bone modulus of the vertebrae are not adversely affected, especially as compared to standard anterior plates and implantation methods. Further, the hyper-angulated approach allows the insertion of prosthetic devices at multiple levels with smaller incisions. Also, in some instances the bone screws and prosthetic devices are easier to access and remove during revision surgeries because of the hyper-angulated approach.

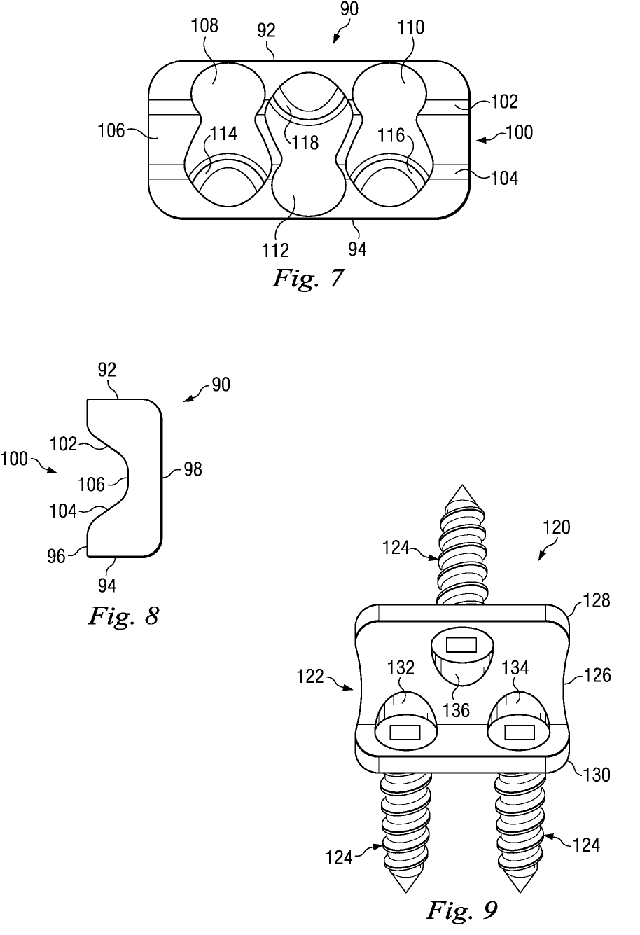

Referring now to FIGS. 7 and 8, shown therein is a prosthetic device 90 according to another embodiment of the present disclosure. In particular, FIG. 7 is a diagrammatic front view of the prosthetic device 90, and FIG. 8 is a diagrammatic side view of the prosthetic device 90. In some aspects the prosthetic device 90 is similar to the prosthetic device 30 described above. The prosthetic device 90 has a generally rectangular profile and includes an upper surface 92 for engaging with an upper vertebra and a lower surface 94 for engaging with a lower vertebra. While the upper and lower surfaces 92 and 94 are shown as being substantially planar in the present embodiment, in other embodiments the surfaces 92 and 94 are contoured to substantially match the endplates of the adjacent vertebra. Further, in some embodiments the surfaces 92 and 94 include bone engagement features or surface textures as described above.

The prosthetic device 90 also includes an anterior surface 96 and an opposing posterior surface 98. In the present embodiment, the anterior surface 96 includes a recessed portion 100. The recessed portion 100 is positioned centrally between the upper and lower surfaces 92 and 94 and extends substantially along the entire length of the prosthetic device. The recessed portion 100 includes tapered surfaces 102 and 104 and planar surface 106. The planar surface 106 extends substantially parallel to the posterior surface 98. The tapered surface 102 extends from an upper portion of the anterior surface 96 and tapers at a constant rate to the planar surface 106. Similarly, the tapered surface 104 extends from a lower portion of the anterior surface 96 and tapers at a constant rate to the planar surface 106. In some instances, the recessed portion 100 allows for the nesting of the heads of the bone screws within the profile of the prosthetic device 90 as defined by the anterior surface 96. In that regard, the bone screw are engaged with the prosthetic device 90 such that the heads of the bone screws do not extend anteriorly beyond the anterior surface 96 and/or outside of the disc space into which the prosthetic device is implanted. Further, in some instances, the recessed portion 100 is utilized by an insertion tool to grasp the prosthetic device 90 during implantation. Further, the recessed portion 100 provides a viewing port in some instances. In that regard, a viewing port is utilized in some instances with longer prosthetic devices 90. Finally, the recessed portion 100 reduces the material density of the prosthetic device 90. Accordingly, in one particular embodiment the titanium density of the prosthetic device 90 is reduced by having the recessed portion 100.

The prosthetic device 90 also includes three openings 108, 110, and 112 extending through the prosthetic device 90 from the anterior surface 96 to the posterior surface 98. Each of the openings 108, 110, and 112 is a generally cylindrical bore sized to receive a bone fixation device for securing the prosthetic device 90 to the adjacent vertebra. In the present embodiment, the opening 112 is substantially centered about a midline or midpoint of the prosthetic device 90, while the openings 108 and 110 are positioned laterally on each side of the opening 112. As discussed above, this orientation of the openings allows the prosthetic device 90 to be utilized adjacent to a spinal level where an implant has been previously implanted, regardless of whether the previous implant is secured to the vertebra with a midline or offset orientation. More specifically, in some instances when the prosthetic device 90 is positioned within the spinal column the opening 112 is utilized to introduce a bone fixation device into the midline of a vertebra, while the openings 108 and 110 are utilized to introduce bone fixation devices in an offset configuration, spaced from the midline of the vertebra. Accordingly, the opening 112 is utilized to secure the prosthetic device 90 to a vertebra that has previously received bone anchors or screws in an offset configuration, while the openings 108 and 110 are utilized to secure the prosthetic device 90 to a vertebra that has previously received bone anchors or screws in a midline configuration.

Each of the openings 108, 110, and 112 of the illustrated embodiment include a seat 114, 116, and 118, respectively. Generally, each of the seats 114, 116, and 118 define a cylindrical bore having a reduced diameter relative to the majority of the corresponding opening 108, 110, and 112. In some instances the seats 114, 116, and 118 are sized and shaped to mate with a head portion of a multi-axial bone screw or other bone anchor. Similar to the openings 44, 46, and 48 of the prosthetic device 30, the openings 108, 110, and 112 extend at an oblique angle with respect to a central plane of the prosthetic device extending parallel to the upper and lower surfaces 92 and 94. Generally, the openings extend at an oblique angle between about 20 degrees and about 70 degrees, and in some instances between about 30 degrees and about 60 degrees relative to the central plane of the prosthetic device. In that regard, the oblique angles are selected to allow insertion of bone fixation devices through the prosthetic device 90 to facilitate engagement of the bone fixation devices with cortical bone of the adjacent vertebrae.

The prosthetic device 90 is suitable for use with hyper-angulated bone fixation devices, and in some embodiments hyper-angulated bone screws. In that regard, the prosthetic device 90 may be positioned entirely within the outer boundaries of the adjacent vertebrae in a no profile orientation when receiving the bone fixation devices. In some instances, at least a portion of the prosthetic device 90, such as a portion of anterior surface 96, extends beyond the anterior boundary of the vertebrae in at least one region when receiving the bone fixation devices. In such instances, the prosthetic device 90 is considered to be implanted in a low profile orientation.

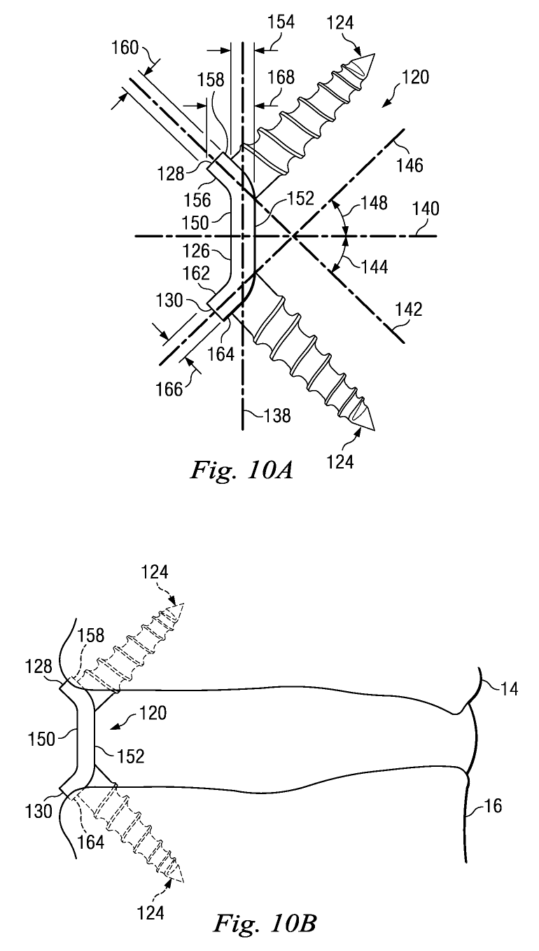

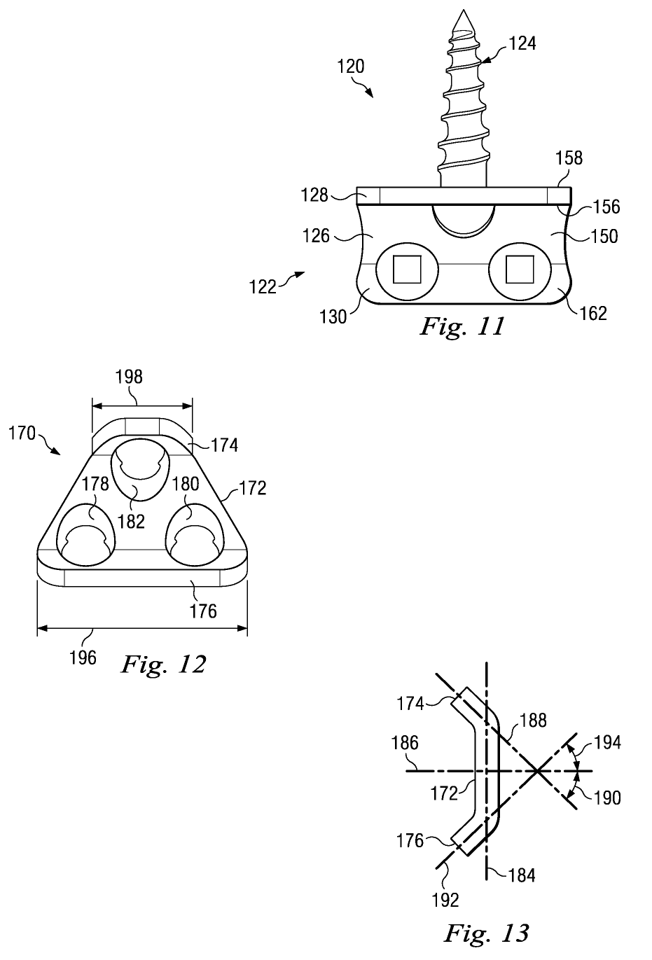

Referring now to FIGS. 9, 10A, 10B, and 11, shown therein is a prosthetic device 120 according to another aspect of the present disclosure. In particular, FIG. 9 is a diagrammatic front view of the prosthetic device 120; FIG. 10A is a diagrammatic side view of the prosthetic device 120; FIG. 10B is a diagrammatic side view of the prosthetic device 120 engaged with a pair of vertebrae 14, 16; and FIG. 11 is a diagrammatic top view of the prosthetic device 120. The prosthetic device 120 comprises a plate 122 that receives three fixation members 124. In the illustrated embodiment, the fixation members 124 are bone screws. The plate 122 includes a central portion 126, an upper portion 128, and a lower portion 130. In the present embodiment, the central, upper, and lower portions 126, 128, 130 are integrally formed such that the plate 122 is monolithic. Openings 132, 134, and 136 extend through the plate 122. More specifically, the openings 132 and 134 extend through at least a part of the lower portion 130, while the opening 136 extends through at least a part of the upper portion 128. In the present embodiment, the openings 132, 134, and 136 are substantially cylindrical bores sized to receive and mate with the fixation members 124.

Referring more specifically to FIG. 10A, a longitudinal axis 138 extends through the plate 122 between the upper portion 128 and the lower portion 130 passing through a midpoint of the central portion 126. An axis 140 extends through the plate 122 and passes through the midpoint of the central portion 126. The axis 140 is substantially perpendicular to the axis 138. In the present embodiment, the central portion 126 extends substantially along and parallel to the axis 138. The upper portion 128 extends along an axis 142 that is at an oblique angle 144 with respect to the axis 138. In the present embodiment the oblique angle 144 is approximately 45 degrees. Generally, the oblique angle 144 is between about 20 degrees and about 70 degrees, and in some instances is between about 30 degrees and about 60 degrees. Similarly, the lower portion 130 extends along an axis 146 that is at an oblique angle 148 with respect to the axis 138. In the present embodiment the oblique angle 148 is approximately 45 degrees. Generally, the oblique angle 148 is between about 20 degrees and about 70 degrees, and in some instances is between about 30 degrees and about 60 degrees.

As shown, the central portion 126 includes an anterior surface 150 and a posterior surface 152. The anterior and posterior surfaces 150, 152 are substantially planar and extend substantially parallel to one another along axis 138. Accordingly, the central portion 126 has a substantially constant thickness 154 as measured between the surfaces 150 and 152. The upper portion 128 includes an anterior surface 156 and a posterior surface 158. The anterior and posterior surfaces 156, 158 are substantially planar and extend substantially parallel to one another along axis 142. Accordingly, the upper portion 128 has a substantially constant thickness 160 as measured between the surfaces 156 and 158. Finally, the lower portion 130 also includes an anterior surface 162 and a posterior surface 164. The anterior and posterior surfaces 162, 164 are substantially planar and extend substantially parallel to one another along axis 146. Accordingly, the lower portion 130 has a substantially constant thickness 166 as measured between the surfaces 162 and 164. In some instances, the thicknesses 154, 160, and 166 are substantially equal to one another such that the plate 122 has a substantially constant thickness. In other embodiments, the thicknesses 154, 160, and 166 of the central portion 126, upper portion 128, and lower portion 130, respectively, vary with respect to one another. In some embodiments, the thicknesses 154, 160, and 166 themselves vary within each of the central portion 126, upper portion 128, and lower portion 130, respectively. In some instances, the particular thicknesses 154, 160, and 166 are determined based on such factors as the material from which the plate is manufactured, the spinal level in which the plate is to be inserted, patient anatomy, and/or other factors.

Referring still to FIG. 10A, in the present embodiment the plate 122 has a maximum profile or width 168 between the posterior surface 152 of the central portion 126 and the anterior most portions of the upper and lower portions 128 and 130. In that regard, in some instances the width 168 is sized such that the plate 122 is utilized in a no profile orientation. In other instances, the width 168 is sized such that the central portion 126 is positioned substantially in a no profile orientation while at least one of the upper and lower portions 128, 130 extends beyond an outer boundary defined by the adjacent vertebra to be in a low profile orientation. In yet other instances, the width 168 and/or the thickness 154 of the central portion 126 are sized such that the central portion is also configured for use in a low profile orientation.

As mentioned above, the openings 132, 134, and 136 extend through the plate 122 such that openings 132 and 134 extend through at least a part of the lower portion 130, while opening 136 extends through at least a part of the upper portion 128. In that regard the openings 132 and 134 extend substantially perpendicular to the anterior and posterior surfaces 162 and 164 of the lower portion 130. Similarly, the opening 136 extends substantially perpendicular to the anterior and posterior surfaces 156 and 158 of the upper portion 128. In the illustrated embodiment the upper and lower portions 128 and 130 extend at angles 144, 148 of approximately 45 degrees with respect to axis 140 such that the upper portion 128 extends substantially perpendicular to the lower portion 130. Accordingly, the opening 136 also extends substantially perpendicular to the openings 132, 134. More specifically, the opening 136 extends substantially perpendicular to axis 142, which in the present embodiment is substantially parallel to axis 146. Similarly, the openings 132, 134 extend substantially perpendicular to axis 146, which is substantially parallel to axis 142 in the present embodiment. In other embodiments, one or more of the openings 132, 134, and 136 extends at an oblique angle with respect to the corresponding upper or lower portion 128, 130 of the plate 122.

Generally, the angles 144, 148 of the upper and lower portions 128, 130 with respect to the axis 140 and/or the angle of the openings with respect to the upper and lower portions 128, 130 is selected to allow insertion of the fixation members 124 through the plate 122 to facilitate engagement of the fixation members with cortical bone of an adjacent vertebra. In that regard, the plate 122 is suitable for use with hyper-angulated bone fixation devices, and in some embodiments hyper-angulated bone screws. In some instances, the hyper-angulated screws facilitate optimal cortical bone purchase or penetration for fixedly securing the plate 122 to the adjacent vertebrae.

For example, referring to FIG. 10B, the prosthetic device 120 is shown secured to vertebra 14 and 16 by fixation members 124 according to one aspect of the present disclosure. The fixation members 124 have been inserted through the openings 132, 134, and 136 (shown in FIG. 9) such that the head portions of the fixation members 124 do not extend beyond the anterior profile of the prosthetic device 120. The fixation members 124 engage the cortical bone of the adjacent vertebrae 14 and 16. In that regard, the hyper-angulated approach allows the fixation members to have greater cortical bone engagement than traditional anterior approaches that extend through the cortical wall of the vertebra. In some instances, the fixation members 124 extend a majority of the way through the vertebrae 14 and 16 and, in some instances, extend substantially across the vertebral bodies without piercing the posterior cortical wall of the vertebrae. Further, similar to earlier embodiments, the prosthetic device 30 is suitable for use in the spinal level adjacent a previously treated spinal level via either a midline approach or an offset approach so as not to disturb the fixation members of a previously implanted device.

Referring now to FIGS. 12 and 13, shown therein is a prosthetic device 170 according to another aspect of the present disclosure. Specifically, FIG. 12 is a diagrammatic front view of the prosthetic device 170, and FIG. 13 is a diagrammatic side view of the prosthetic device 170. In some aspects, the prosthetic device 170 is similar to the prosthetic device 120 described above and, therefore, not all aspects of the device 170 will be described in detail. Generally, when view from the front or anterior side, the device 170 has a generally triangular profile rather than the generally rectangular profile of the prosthetic device 120. The prosthetic device 170 comprises a central portion 172, an upper portion 174, and a lower portion 176. In the present embodiment, the central, upper, and lower portions 172, 174, 176 are integrally formed such that the prosthetic device 170 is monolithic. Openings 178, 180, and 182 extend through the device 170. More specifically, the openings 178 and 180 extend through at least a part of the lower portion 176, while the opening 182 extends through at least a part of the upper portion 174.

Referring more specifically to FIG. 13, a longitudinal axis 184 extends through the device 170 between the upper portion 174 and the lower portion 176 passing through a midpoint of the central portion 172. An axis 186 extends through the device 170 and passes through the midpoint of the central portion 172. The axis 186 is substantially perpendicular to the axis 184. In the present embodiment, the central portion 126 extends substantially along and parallel to the axis 184. The upper portion 174 extends along an axis 188 that is at an oblique angle 190 with respect to the axis 186. The lower portion 176 extends along an axis 192 that is at an oblique angle 194 with respect to the axis 186. The oblique angles 190 and 194 have similar ranges to those described above for angles 144 and 148.

Referring more specifically to FIG. 12, the lower portion 176 has a maximum width 196 between its lateral edges, while the upper portion 174 has a maximum width 198 between its lateral edges. The maximum width 196 of the lower portion 176 is greater than the maximum width 198 of the upper portion 174. In that regard, the maximum width 196 of the lower portion 176 is generally between about 4 mm and about 15 mm. Whereas the maximum width 198 of the upper portion 174 is generally between about 8 mm and about 25 mm. In that regard, the central portion 172 tapers between the upper portion 174 and the lower portion 176 such that the device 120 has a generally triangular profile as viewed from the front or anterior side of the device. In the present embodiment, the central portion 172 and the device 120 as a whole have a substantially constant taper between the lower portion 176 and the upper portion 174. In other embodiments, the taper is not constant and varies along the height of the device.

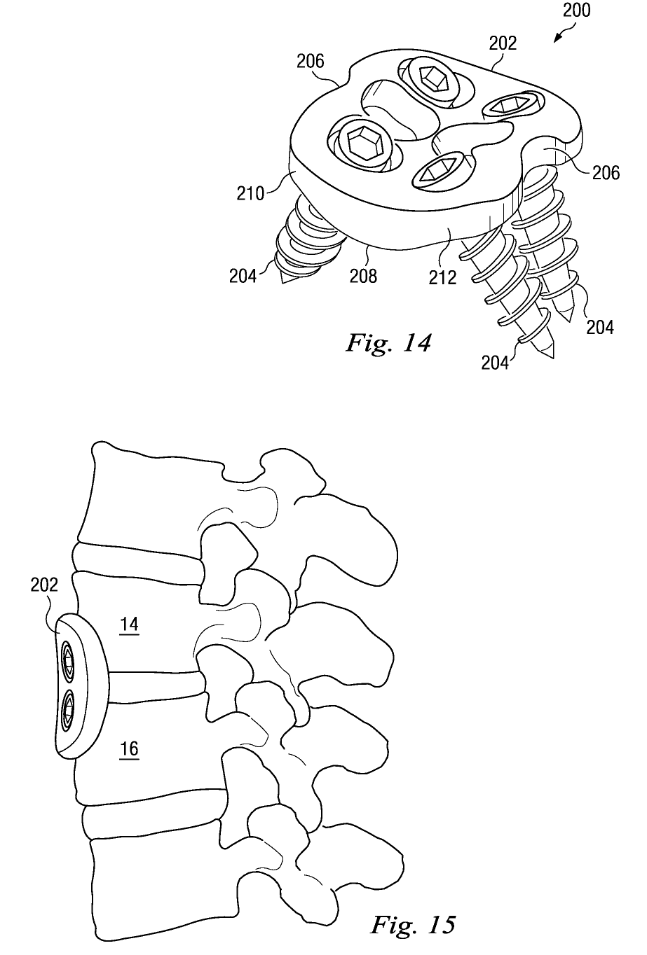

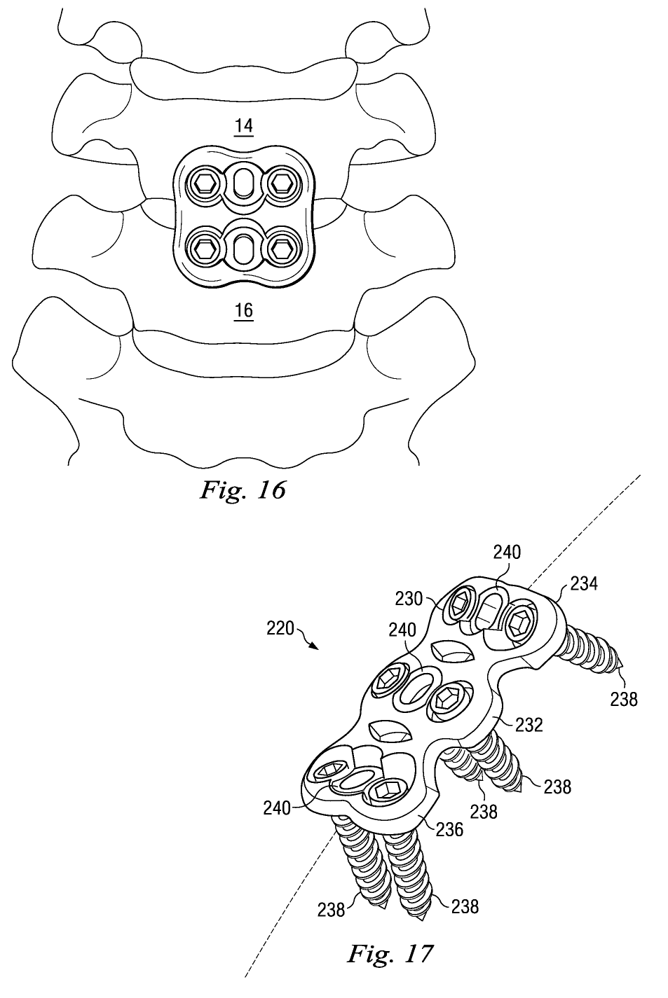

Referring now to FIGS. 14-16, shown therein is a prosthetic device 200 according to another embodiment of the present disclosure. Specifically, FIG. 14 is a diagrammatic perspective view of the prosthetic device 200; FIG. 15 is a diagrammatic side view of the prosthetic device 200 engaged with vertebrae 14 and 16; and FIG. 16 is a diagrammatic front view of the prosthetic device 200 engaged with the vertebrae 14 and 16. The prosthetic device 200 includes a plate portion 202 with openings for receiving four bone anchors 204, which are secured to the plate portion 202 by locking members 206. In that regard, the plate portion 202 is a low-profile short plate sized to be positioned between vertebra of a single spinal level. The plate portion 202 includes a central section 208, an upper section 210, and a lower section 212. In the present embodiment, the central section 208 has an increased thickness relative to the upper and lower sections 210, 212. The increased thickness of the central section 208 is sized to at least partially extend within the outer boundaries of the vertebrae 14, 16 as shown in FIG. 15. In that regard, the taper or contour of the plate 202 is sized and shaped to match the corresponding anatomical features of the vertebrae 14, 16 in some instances. The upper and lower sections 208, 210 each include a pair of openings extending therethrough for receiving the bone anchors 204 in an hyper-angulated orientation. In that regard, in some instances the openings are formed through the plate portion 202 at an oblique angle with respect to a central axis of the plate, similar to the openings of the other prosthetic devices described above.

In addition to the openings for receiving the bone anchors, the upper and lower sections 208, 210 include a recessed portion for receiving locking member 206 for securing the bone anchors 204 to the plate portion 202. Generally, any suitable locking member 206 is utilized to secure the bone anchors 204 in place. In some instances, the locking members 206 are similar to those described in U.S. Pat. No. 7,169,150 titled “NON-METALLIC ORTHOPEDIC PLATE”, which is hereby incorporated by reference in its entirety. In other instances, the locking members comprise a single Nitinol wire. Further, while not explicitly described with respect to some embodiments of the present disclosure it is understood that in some instances locking members are utilized with the other prosthetic devices of the present disclosure to secure the bone fixation members to the prosthetic devices.

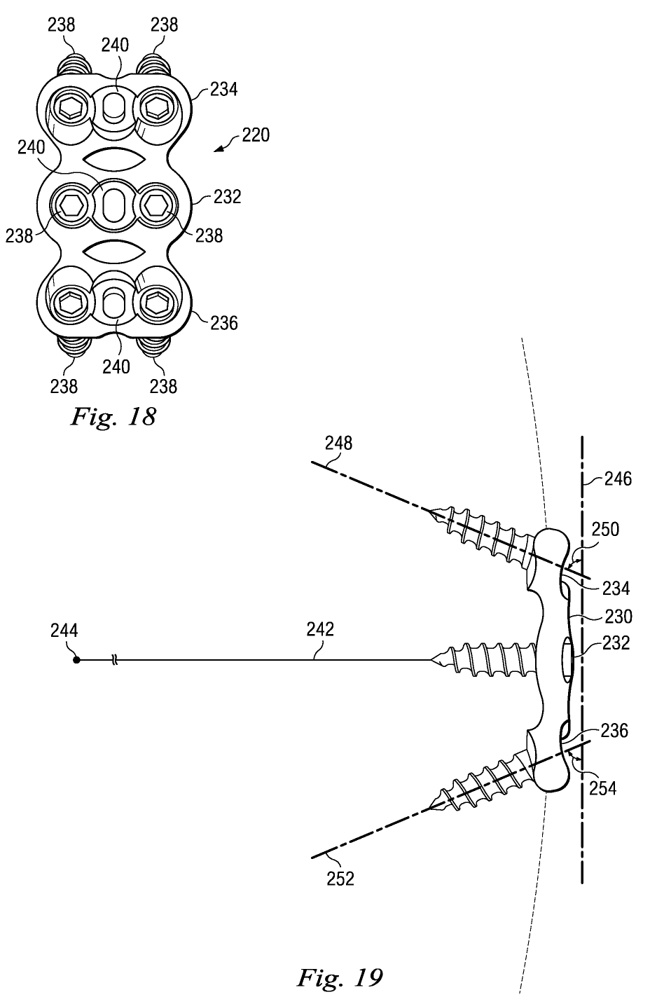

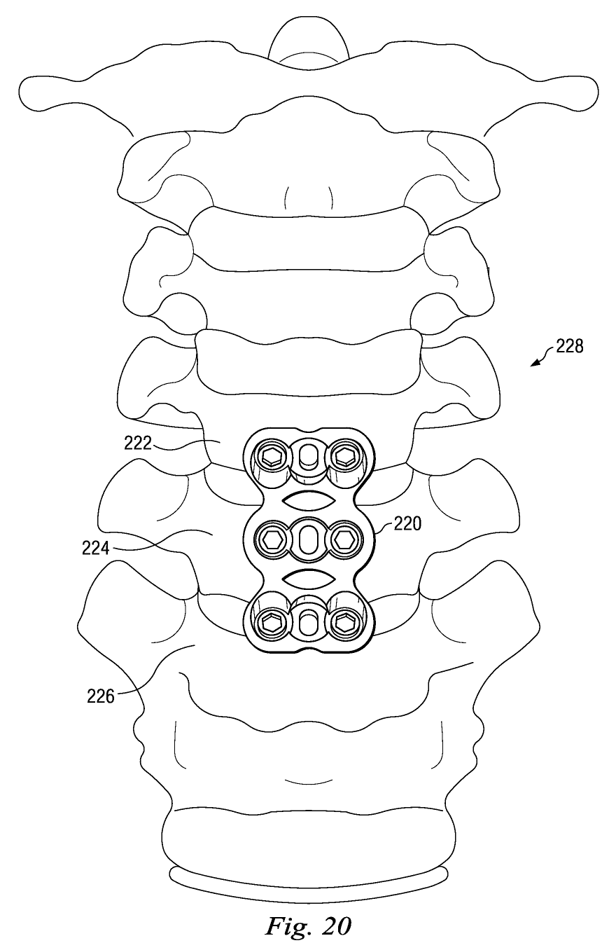

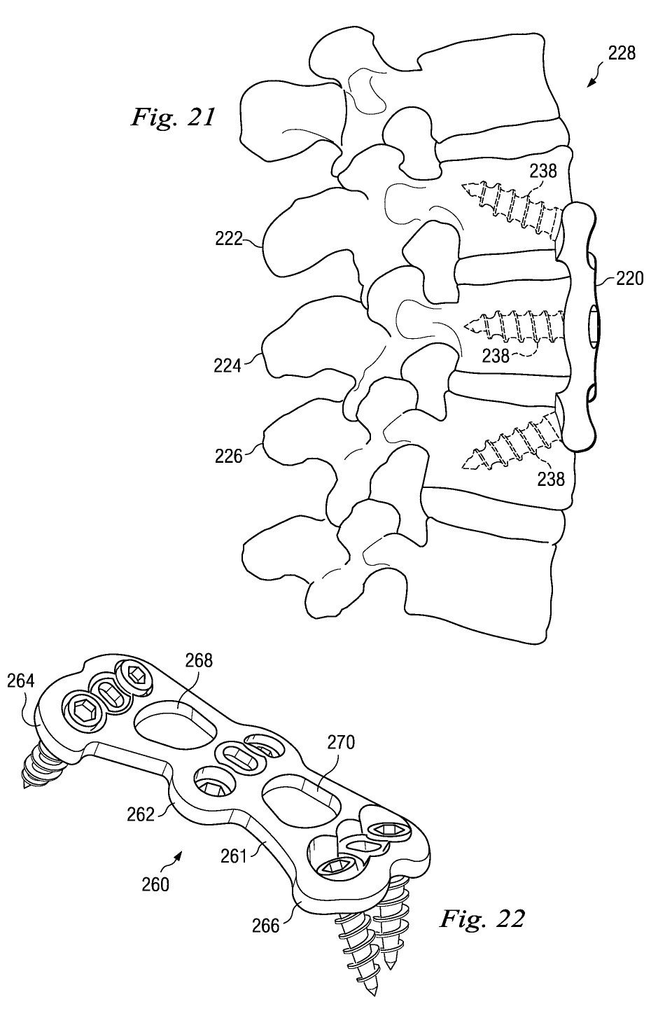

Referring now to FIGS. 17-22, shown therein is a prosthetic device 220 according to another aspect of the present disclosure. Specifically, FIG. 17 is a diagrammatic perspective view of the prosthetic device 220; FIG. 18 is a diagrammatic front view of the prosthetic device 220; FIG. 19 is a diagrammatic side view of the prosthetic device 220; FIG. 20 is a diagrammatic front view of the prosthetic device 220 engaged with three vertebrae 222, 224, and 226 of a spinal column 228; and FIG. 21 is a diagrammatic side view of the prosthetic device 220 engaged with the vertebrae 222, 224, and 226. As shown, the prosthetic device 220 includes a plate 230 having a middle region 232, an upper region 234, and a lower region 236. Each of the regions 232, 234, and 236 receives a pair of bone screws 238 that are secured to the plate 230 by locking mechanisms 240.

Referring more specifically to FIG. 19, the plate 230 is generally curved along its length between the upper region 234 and the lower region 238. In the present embodiment, the plate 230 has a radius of curvature 242 from a center point 244. Generally, the radius of curvature 242 is selected to match the corresponding curvature of the portion of the spinal column into which the plate 230 is to be implanted. In some instances the radius of curvature 242 is between about 170 mm and about 200 mm. In other instances, the plate 230 is substantially planar such that he radius of curvature is approximately infinite. In other instances, the plate 230 comprises a plurality of planar portions that together generally define a curvature along the length of the plate.

As illustrated, an axis 246 extends substantially perpendicular to an axis coincident with the radius of curvature 242 passing through the center point 244 and a midpoint of the plate 230. In that regard, the axis 246 is a tangent to the arc defined by the radius of curvature 242 at the midpoint of the plate 230. The bone screws 238 received by the middle region 232 extend substantially parallel to the axis coincident with the radius of curvature 242. Accordingly, the bone screws 238 received by the middle region 232 extend substantially perpendicular to the axis 246. Further, the bone screws 238 received by the upper region 234 extend along an axis 248, which is at an oblique angle 250 relative to the axis 246. Similarly, the bone screws 238 received by the lower region 236 extend along an axis 252, which is at an oblique angle 254 relative to the axis 246. Generally, the angles 250 and 254 are between about 20 degrees and about 70 degrees, and in some instances are between about 30 degrees and about 60 degrees. In the illustrated embodiment, each of the angles 250 and 254 is approximately 20 degrees. In this manner the plate 230 is shaped to receive the bone screws 238 through the upper and lower regions 234, 236 in a hyper-angulated orientation. In that regard, in some instances the bores or openings of the upper and lower regions 234, 236 that receive the bone screws 238 extend substantially parallel to the axes 248 and 250.

Referring more specifically to FIGS. 20 and 21, the plate 230 is shown attached to the spinal column 228 and, more particularly, vertebra 222, 224, and 226. In that regard, the plate 230 is sized such that the upper region 234 extends over only the lower most portion of vertebra 222, while the lower region 236 extends over only the upper most portion of vertebra 226. Accordingly, in some instances the plate 230 may be utilized adjacent spinal levels where previous spinal plate or other prosthetic device has been implanted without interfering with the previously implanted device. Further, while the upper and lower regions 234, 236 only extend over a portion of the vertebra 222, 226, respectively, the angled orientation of the bone screws 238 passing through each of the regions 234, 236 allows the bone screws to penetrate the cortical bone of the vertebra 222, 226 to provide a secure attachment for the plate 230. As shown, the prosthetic device 220 is utilized to stabilize two spinal levels in some instances.

Referring now to FIG. 22, shown therein is a diagrammatic perspective view of a prosthetic device 260 according to another embodiment of the present disclosure. Generally, the prosthetic device 260 is similar to the prosthetic device 220 described above. In that regard, the prosthetic device 260 includes a plate 261 having a central portion 262, an upper portion 264, and a lower portion 266. The upper and lower portions 264, 266 are configured to receive bone anchors or screws in a hyper-angulated orientation. However, the prosthetic device 260 includes a window 268 between the central portion 262 and the upper portion 264 and a window 270 between the central portion 262 and the lower portion 266. In some instances, the windows 268 and 270 allow monitoring of fusion between the vertebrae using medical imaging. For example, in some instances the windows 268, 270 allow standard radiographic techniques to visualize the bone ingrowth and/or fusion occurring between the vertebrae.

Generally, the prosthetic devices, plates, and fixation members of the present disclosure are constructed of any suitable medical grade material. In that regard, desired features of the particular component, such as strength, flexibility, radiopaque/radiolucent, hardness, weight, wear resistance, and/or other characteristics, are considered in selecting the suitable material. Suitable biocompatible materials include metals, ceramics, polymers, and combinations thereof. For example, in some embodiments metals such as cobalt-chromium alloys, titanium alloys, nickel titanium alloys, and stainless steel alloys are suitable. In other embodiments, ceramic materials such as aluminum oxide or alumina, zirconium oxide or zirconia, compact of particulate diamond, or pyrolytic carbon are suitable. In yet other embodiments polymer materials are used, including members of the polyaryletherketone (PAEK) family such as polyetheretherketone (PEEK), carbon-reinforced PEEK, other modified PEEK materials, or polyetherketoneketone (PEKK); polysulfone; polyetherimide; polyimide; ultra-high molecular weight polyethylene (UHMWPE); cross-linked UHMWPE; silicon, polycarbonate urethanes, and nano-material treated polymers. In some instances, the materials are imbedded with one or more radiographic markers.

In some embodiments, the devices or individual components are constructed of bone or other tissue materials. Tissue materials include, but are not limited to, synthetic or natural autograft, allograft or xenograft, and may be resorbable or non-resorbable in nature. Examples of tissue materials include, but are not limited to, hard tissues, connective tissues, demineralized bone matrix and combinations thereof. Examples of resorbable materials that are used in some instances include, but are not limited to, polylactide, polyglycolide, tyrosine-derived polycarbonate, polyanhydride, polyorthoester, polyphosphazene, calcium phosphate, hydroxyapatite, bioactive glass, PLLA, PLDA, and combinations thereof. Further still, in some circumstances an interior portion or cavity of the prosthetic devices is packed with a suitable osteogenic material, bone morphogenetic proteins, or therapeutic composition to encourage bone growth. Osteogenic materials include, without limitation, autograft, allograft, xenograft, demineralized bone, synthetic and natural bone graft substitutes, such as bioceramics and polymers, and osteoinductive factors.

While specific embodiments have been illustrated and described in detail in the drawings and foregoing description, this is to be considered illustrative and not restrictive in character. It is understood that one of ordinary skill will be able to effect various alterations, substitutions of equivalents, and other modifications without departing from the concepts disclosed herein.

We claim:

1. A spinal plate for positioning between a first vertebra and a second vertebra, the spinal plate comprising:
a generally rectangular body portion comprising:
a first elongated engagement surface for fixedly engaging the first vertebra;
a second elongated engagement surface opposite the first engagement surface for fixedly engaging the second vertebra, the second engagement surface extending substantially parallel to the first engagement surface, the first and second engagement surfaces separated by a first height;
a first axis extending substantially perpendicular to the first and second engagement surfaces;
a first sidewall extending between and substantially perpendicular to the first and second engagement surfaces;
a second sidewall extending between and substantially perpendicular to the first and second engagement surfaces opposite the first sidewall, the second sidewall extending substantially parallel to the first sidewall, the first and second sidewalls separated by a first width, the first height being greater than the first width;
a first substantially cylindrical bore extending from the first sidewall to the second sidewall through the body portion at an oblique angle of at least 30 degrees with respect to the first axis, the first bore sized to receive and mate with a bone fixation device for securing the body portion to the first vertebra.

2. The spinal plate of claim 1, wherein the first height is at least twice the first width.

3. The spinal plate of claim 2, wherein the first engagement surface includes at least one fixation element for penetrating an endplate of the first vertebrae.

4. The spinal plate of claim 1, wherein the first bore extends from a portion of the first sidewall adjacent the second engagement surface to a portion of the second sidewall adjacent the first engagement surface.

5. The spinal plate of claim 4, further comprising a second substantially cylindrical bore extending from the first sidewall to the second sidewall through the body portion at an oblique angle of at least 30 degrees with respect to the first axis, the second bore sized to receive and mate with a bone fixation device for securing the body portion to the second vertebra.

6. The spinal plate of claim 5, wherein the second bore extends from a portion of the first sidewall adjacent the first engagement surface to a portion of the second sidewall adjacent the second engagement surface.

7. The spinal plate of claim 6, further comprising a third substantially cylindrical bore extending from the first sidewall to the second sidewall through the body portion at an oblique angle of at least 30 degrees with respect to the first axis and substantially parallel to the first bore, the third bore sized to receive and mate with a bone fixation device for securing the body portion to the first vertebra, the third bore extending from a portion of the first sidewall adjacent the second engagement surface to a portion of the second sidewall adjacent the first engagement surface.

8. The spinal plate of claim 7, wherein the body portion further comprises:
a first end portion extending between the first engagement surface, the second engagement surface, the first sidewall, and the second sidewall, and
a second end portion opposite the first end portion, the second end portion extending between the first engagement surface, the second engagement surface, the first sidewall, and the second sidewall;
wherein the first bore is positioned towards the first end portion, the third bore is positioned towards the second end portion, and the second bore is positioned between the first and third bores.

9. The spinal plate of claim 8, wherein the first, second, and third bores are equally spaced along a length of the body portion between the first end portion and the second end portion.

10. The spinal plate of claim 8, wherein the second bore at least partially intersects the first and third bores.

11. A spinal implant for stabilizing a pair of adjacent vertebrae without penetrating a sidewall of the vertebrae, the spinal implant comprising:
a central portion extending along a first plane;
a first engagement portion extending from an upper part of the central portion, the first engagement portion extending along a second plane, the second plane being at a first oblique angle with respect to the first plane;
a first opening extending through the first engagement portion substantially perpendicular to the second plane, the first opening sized and shaped to receive and mate with a first bone fixation device for securing the first engagement portion to one of the adjacent vertebrae;
a second engagement portion extending from a lower part of the central portion, the second engagement portion extending along a third plane, the third plane being at a second oblique angle with respect to the first plane and substantially perpendicular to the second plane;
a second opening extending through the second engagement portion substantially perpendicular to the third plane, the second opening sized and shaped to receive and mate with a second bone fixation device for securing the second engagement portion to the other of the adjacent vertebrae.

12. The spinal implant of claim 11, wherein the central portion has a central thickness less than ⅓ the length of the disc space between the adjacent vertebrae.

13. The spinal implant of claim 12, wherein the central portion has a central height less than the height of the disc space between the adjacent vertebrae such that the central portion is positionable entirely between the adjacent vertebrae.

14. The spinal implant of claim 13, further comprising a third opening extending through the first engagement portion substantially perpendicular to the second plane and substantially parallel to the first opening, the third opening sized and shaped to receive and mate with a third bone fixation device for securing the first engagement portion to one of the adjacent vertebrae.

15. The spinal implant of claim 14, wherein the first engagement portion has a first width, and wherein the second engagement portion has a second width, the second width being less than or equal to the first width.

16. The spinal implant of claim 15, wherein the second width is less than the first width, and wherein the implant has a substantially constant taper from first width of the first engagement portion through the central portion and to the second width of the second engagement portion to define a generally triangular profile.

17. The spinal implant of claim 15, wherein the first oblique angle is approximately 45 degrees.

18. The spinal implant of claim 17, wherein the central portion, the first engagement portion, and the second engagement portion have substantially equal thicknesses.

19. A method of stabilizing a first vertebra and a second vertebra adjacent to a previously stabilized spinal level that includes the first vertebra, the method comprising:
providing a prosthetic device sized to fit substantially within a disc space between the first and second vertebra;
gaining access to the disc space;
inserting the prosthetic device into the disc space such that the prosthetic device is in at least a low profile orientation with respect to the first and second vertebra and such that the prosthetic device extends within the disc space less than ⅓ of the length of the vertebral bodies of the first and second vertebra;
extending a first bone anchor through a first bore in the prosthetic device and engaging the first bone anchor with an endplate of the first vertebra such that the first bone anchor extends at an angle of approximately 45 degrees relative to a central axis of the prosthetic device; and
extending a second bone anchor through a second bore in the prosthetic device and engaging the second bone anchor with an endplate of the second vertebra such that the second bone anchor extends at an angle of approximately 45 degrees relative to the central axis the prosthetic device, such that the first bone anchor and the second bone anchor extend substantially perpendicular to one another.

20. The method of claim 19, wherein inserting the prosthetic device comprises positioning the prosthetic device within the disc space such that the prosthetic device is in a no profile orientation with respect to the first and second vertebra.
